(19) United States
(12) Patent Application Publication
(10) Pub. No.: US 2008/0057015 A1
(43) Pub. Date: Mar. 6, 2008

(54) Hair care compositions, methods, and articles of commerce that can help maintain a longer lasting hair style appearance

(76) Inventors: John Erich Oblong, Loveland, OH (US); Robert Scott Youngquist, Mason, OH (US); Thomas Larry Dawson JR., Hamilton, OH (US); Nikki Elizabeth Annunziata, Harrison, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/895,127

(22) Filed: Aug. 23, 2007

Related U.S. Application Data
(60) Provisional application No. 60/841,094, filed on Aug. 30, 2006.

(51) Int. Cl. A61K 8/97; A61K 8/00; G06Q 30/00

(52) U.S. Cl. 424/70.2; 424/74; 705/14

(57) ABSTRACT

Hair care compositions, methods, and articles of commerce that can help maintain a longer lasting hair style appearance, including coloring and grooming. Such compositions can be applied to any areas where maintenance of longer lasting hair style appearance is desired, such as the scalp, neck, and/or beard. The present invention also relates to methods of marketing such compositions.

### CROSS REFERENCE TO RELATED APPLICATION

This application claims the benefit of U.S. Provisional Application Ser. No. 60/841,094 filed Aug. 30, 2006.

### FIELD

The present invention relates to hair care compositions, methods, and articles of commerce that can maintain a longer lasting hair style appearance, including coloration and grooming on the head, neck, and face of consumers. The present invention also relates to methods of marketing such compositions.

### BACKGROUND

Because human hair continues to grow, consumers must exert a great deal of continuing effort in order to maintain their desired hair style. These efforts can include, for example, cutting to maintain length, re-coloring to cover the roots of color-treated hair, and/or re-perming to curl the hair that has grown out from the roots. This effort is expended by both female and male consumers.

In addition to the cost and inconvenience associated with this on-going hair style maintenance, consumers can also experience a less than desired appearance in between maintenance events, as the hair grows to expose untreated roots and/or to create a longer than desired hair style.

Accordingly, there is a need to provide consumers with a way to maintain a longer lasting hair style appearance, thus reducing the frequency of hair style maintenance needed in order to maintain their hair style.

### SUMMARY

The present invention relates to hair care compositions, methods, and articles of commerce that can help maintain a longer lasting hair style appearance, thus reducing the frequency of hair style maintenance needed in order to maintain their hair style. The present invention also relates to methods of marketing.

In one aspect, a method comprises topically applying a hair care composition comprising an effective amount of a hair growth inhibiting agent to the scalp of a mammal for the purpose of helping to maintain a longer lasting hair style appearance. In another embodiment, a method comprises topically applying a hair care composition comprising an effective amount of a hair growth inhibiting agent to the neck of a mammal for the purpose of helping to maintain a longer lasting hair style appearance. In another embodiment, a method comprises topically applying a hair care composition comprising an effective amount of a hair growth inhibiting agent to the face of a mammal for the purpose of helping to maintain a longer lasting hair style appearance, by reducing the frequency of hair coloration maintenance and/or maintaining a longer lasting style. In another embodiment, a method comprises topically applying a hair care composition comprising an effective amount of a hair growth inhibiting agent to the scalp and/or face of a mammal for the purpose of helping to reduce the frequency of hair coloration maintenance, such as with temporary and/or permanent hair dyes.

In particular embodiments, the type of hair style maintenance is selected from the group consisting of hair coloring (e.g., for the purpose of retarding the growth and thus the appearance of non-color-treated hair roots), hair cutting and/or trimming (e.g., for the purpose of restoring the length of hair to the length of a prior cut and/or trim), permanent hair curling (e.g., for the purpose of curling the length of hair that has grown subsequent to a prior permanent wave treatment), permanent hair waving, permanent hair relaxing, permanent hair straightening (e.g., to straighten the length of hair that has grown subsequent to a prior straightening treatment), and combinations thereof. Accordingly, the invention herein can be used to help retain style benefits between haircuts and/or trims, as well as for retention of chemical and/or mechanical treatments (e.g., perms, waves, relaxants, straightening, or coloring), and combinations thereof. This includes application to the neck, face, eyebrows, and scalp hair.

These and other features, aspects, and advantages of the present invention will become evident to those skilled in the art from a reading of the present disclosure.

### DETAILED DESCRIPTION

While the specification concludes with the claims particularly pointing and distinctly claiming the invention, it is believed that the present invention will be better understood from the following description.

In the past, various compositions comprising hair growth inhibiting agents have been applied to body areas where hair is not desired, in an effort to eliminate, decrease, and/or slow the growth of unwanted hair. However, applicants are not aware of any methods or teaching to use such hair growth inhibition agent compositions for the purpose of slowing the growth of hair in areas of the body (e.g., the human head) where hair is desired, such that the desired hair style can be maintained for longer periods between hair style maintenance events. Thus, the present invention provides methods for reducing the frequency of hair style maintenance, by helping to maintain a longer lasting hair style appearance.

Because consumers are not familiar with the use of hair growth inhibiting agents for the purpose of maintaining a longer lasting hair style appearance, the present invention also provides methods of marketing that can be advantageously used to help potential consumers appreciate the benefits that they can derive from such a product and/or its method of use. Furthermore, a method of marketing a first composition by comparing it to a second composition that comprises a hair growth inhibition agent is also provided.

All percentages, parts and ratios are based upon the total weight of the hair care compositions of the present invention and all measurements made are at 25° C., unless otherwise specified. All such weights as they pertain to listed ingredients are based on the active level and, therefore, do not include carriers or by-products that may be included in commercially available materials, unless otherwise specified.

As used herein, the term “hair care compositions” are compositions that are applied to the hair and/or the skin underneath the hair, including compositions used to treat or care for the hair. Products contemplated by the phrase “hair care composition” include, but are not limited to liquids, creams, wipes, hair conditioners (rinse-off and leave-on), hair tonics, shampoos, hair colorants, mousses, propellant lotions, emulsions, shave gels, after-shave tonics and lotions, temporary beard hair dyes, and the like.

“Hair growth inhibiting agent” includes any material that can reduce, inhibit, attenuate, or diminish mammalian hair growth.

“Maintain a longer lasting hair style appearance” means retain hair style appearance for a longer period of time. This is demonstrated by a reduction of hair growth when a composition of the present invention is used for a result-effective period of time.

“Reduction of hair growth” as referenced herein, is demonstrated when the growth rate of hair is reduced by a statistically significant amount.

As used herein, the terms “hair style maintenance” and “hair style maintenance event” are used interchangeably to mean a hair styling procedure performed to restore a hair style that has been altered by growth of hair that was previously styled.

“Mammalian hair,” as referenced herein, includes hair on any part of the body of a mammal, and can include but is not limited to facial, cranial, or body hair. For instance, it can include hair on the scalp, head, neck, beard, moustache, eyebrows and sideburns hair.

The term “topical application,” as used herein, means to apply or spread the compositions of the present invention onto the surface of the keratinous tissue.

The term “dermatologically-acceptable,” as used herein, means that the compositions or components thereof so described are suitable for use in contact with mammalian keratinous tissue without undue toxicity, incompatibility, instability, allergic response, and the like.

The term “result-effective period of time,” as used herein, means a period of time sufficient to reduce the growth rate of hair by a statistically significant amount.

The term “effective amount,” as used herein, means an amount of a compound or composition sufficient to reduce the growth rate of hair by a statistically significant amount.

The term “safe and effective amount,” as used herein, means an amount of a compound or composition sufficient to reduce the growth rate of hair by a statistically significant amount, but low enough to avoid serious side effects, i.e., to provide a reasonable benefit to risk ratio, within the scope of sound judgment of the skilled artisan.

The term “ambient conditions,” as used herein, refers to surrounding conditions under about one atmosphere of pressure, at about 50% relative humidity, and at about 25° C., unless otherwise specified.

I. Hair Care Compositions

In one aspect, the present invention provides hair care compositions that can be used to help maintain a longer lasting hair style appearance. In one embodiment, the hair care composition comprises a hair growth inhibiting agent. In another embodiment, the hair care composition comprises two or more hair growth inhibiting agents. Preferably, the hair growth inhibiting agent(s) is present in an effective amount, more preferably in an effective amount. As used herein, the singular term “hair growth inhibiting agent” is broad enough to include one or a combination of more than one hair growth inhibition agent. Optionally, the hair care compositions can comprise a dermatologically-acceptable carrier and/or any desired suitable optional ingredients.

The hair care compositions can be in any suitable form, such as a liquid, cream, shampoo, conditioner, tonic, etc.

Any suitable hair growth inhibition agent can be used herein, preferably in an effective amount, more preferably in an effective amount. In a particular embodiment, the hair growth inhibition agent can be selected from the group consisting of butylated hydroxytolune, butylated hydroxyanisole, hexamidine, hexyl isobutyrate, menthyl anthranilate, methofuran, 3-butylidenepthalide, glyceryl dilaurate, hexanediol, panthenol, agmatine, aminoguanidine, phenyl butyl nitrone and other spin traps, ethoxyquin, cetyl pyridinium chloride, green tea extract, catechins, phytosterols, ursolic acid, plant extracts, plant extract compounds, 3-butylidenepthalide, its salts, its derivatives, and mixtures thereof and combinations thereof. Particular hair growth inhibition agents are described in more detail below.

A. Butylated Hydroxytoluene (BHT) and Butylated Hydroxyanisole (BHA)

The hair care compositions of the present invention can comprise an effective amount of BHT and/or BHA. The BHT useful herein can be described by the general structure: 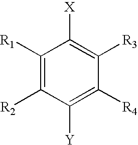
where in X is selected from the group consisting of OH and SH;
Y is selected from the group consisting of H, OH, OR₅, COOR₅, alkyl, cycloalkyl, heteroalkyl, heterocycloalkyl, aromatic, heteroaromatic, carboxamido, sulfonamido, carbamate, urea, and trialkylsilyl;
R₁, R₂, R₃, R₄ are selected from the group consisting of alkyl, cycloalkyl, heteroalkyl, heterocycloalkyl, aromatic, heteroaromatic, OR₅, carboxamido, sulfonamido, formyl, acyl, carboxyl, carboxylate, carbamate, urea, trialkylsilyl, hydroxyl, and hydrogen;
R₅ is selected from the group consisting of alkyl, cycloalkyl, heteroalkyl, heterocycloalkyl, aromatic, heteroaromatic, trialkylsilyl, acyl, and hydrogen.

BHA and BHT can be purchased from various suppliers, including Eastman Chemical (Kingsport, Tenn.), Alfa Chemical (Kings Point, N.Y.), and Shell Chemical Company (Houston, Tex.).

In one embodiment, BHT and/or BHA can be present in an amount of from about 0.0001% to about 50%, in another embodiment from about 0.001% to about 10%, in still another embodiment from about 0.01% to about 5%, and in yet another embodiment from about 0.1% to about 1% by weight of the composition.

B. Hexamidine

The topical composition of the present invention may comprise an effective amount of hexamidine, its salt, and/or derivatives thereof. More preferably, the hexamidine is hexamidine isethionate. As used herein, “hexamidine” includes any isomers and tautomers of such and is commercially available as hexamidine isethionate under the tradename Elastab® HP100 from Laboratoires Serobiologiques (Pulnoy, France).

In one composition of the present invention, hexamidine comprises from about 0.0001% to about 20% by weight of the composition, in another embodiment from about 0.001% to about 10%, in yet another embodiment from about 0.01% to about 5%, and in yet another embodiment from about 0.1% to about 2%.

C. Hexyl Isobutyrate

The hair care compositions of the present invention may comprise an effective amount of hexyl isobutyrate, its salt, and/or derivatives thereof. Hexyl isobutyrate may be present in an amount of from about 0.0001% to about 50% by weight of the composition, in another embodiment from about 0.001% to about 10%, in yet another embodiment from about 0.01% to about 5%, and in still another embodiment from about 0.1% to about 2%.

D. Menthyl Anthranilate

The hair care compositions of the present invention may comprise an effective amount of menthyl anthranilate, its salt, and/or derivatives thereof. Menthyl anthranilate is commercially available from Phoenix Aromas & Essential Oils, Inc. (Norwood, N.J.) and Alzo International (Sayreville, N.J.).

Menthyl anthranilate may be present in an amount of from about 0.0001% to about 50% by weight of the composition, in another embodiment from about 0.001% to about 20%, in another embodiment from about 0.01% to about 10%, and in yet another embodiment from about 0.1% to about 5%.

E. Methofuran

The hair care compositions of the present invention may comprise an effective amount of methofuran, its salt, and/or derivatives thereof. Methofuran is commercially available from Aldrich Chemical Company (Milwaukee, Wis.), and Sigma Chemical Company (St. Louis, Mo.).

Methofuran may be present in an amount of from about 0.0001% to about 50% by weight of the composition, in another embodiment from about 0.001% to about 20%, in still another embodiment from about 0.01% to about 10%, and in yet another embodiment from about 0.1% to about 5%.

F. Agmatine

The topical composition of the present invention can comprise an effective amount of agmatine. Agmatine useful herein can be described by the general structure: 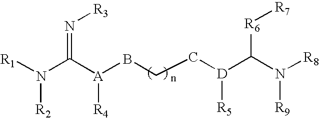
R₁, R₂, R₃, R₄, R₅, R₆, R₇, R₈, R₉=alkyl, cycloalkyl, heteroalkyl, heterocycloalkyl, aromatic, heteroaromatic, carboxamido, sulfonamido, carboxyl, carboxylate, carbamate, urea, sulfate, phosphate, silyl, acyl, amino acid, polypeptide, imino, hydrogen, nitro, nitrogen, oxygen, sulfur, phosphorus, nil.
A, D=carbon, nitrogen, aromatic, heteroaromatic, hydrogen, nil.
B, C=nitrogen, oxygen, sulfur, phosphorus, hydrogen, nil.
n=0 to 20.

In the composition of the present invention, agmatine can comprise from about 0.0001% to about 99.99% by weight of the composition, in a different embodiment from about 0.001% to about 10%, in another embodiment from about 0.01% to about 5%, and in still another embodiment from about 0.1% to about 2.5%.

G. 3-butylidenepthalide

The hair care compositions of the present invention may comprise an effective amount of 3-butylidenepthalide, its salt, and/or derivatives thereof. 3-butylidenepthalide may be present in an amount of from about 0.0001% to about 50% by weight of the composition, in another embodiment from about 0.001% to about 20%, in a different embodiment from about 0.01% to about 10%, and in yet another embodiment from about 0.1% to about 5%.

H. Cetyl Pyridinium Chloride

The hair care compositions of the present invention may comprise an effective amount of cetyl pyridinium chloride and/or its alternate forms. Alternate forms of cetyl pyridinium chloride include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms (typically methyl or ethyl groups). Dodecyl trimethyl ammonium bromide, tetradecylpyridinium chloride, domiphenbromide, N-tetradecyl-4-ethyl pyridinium chloride, dodecyl dimethyl (2-phenoxyethyl) ammonium bromide, benzyl dimethylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium agents. Other compounds that may be included are bis-4-(R-amino)-1-pyridinium alkanes as disclosed in U.S. Pat. No. 4,206,215.

Cetyl pyridinium chloride may be present in an amount of from about 0.0001% to about 50% by weight of the composition, in another embodiment from about 0.001% to about 5%, in still another embodiment from about 0.01% to about 2%, and in a different embodiment from about 0.05% to about 1%.

I. Green Tea Extract and Catechins

The hair care compositions of the present invention may comprise an effective amount of one or more catechin compounds, including but not limited to those selected from the group consisting of green tea extracts, catechin, epicatechin, epigallocatechin, epicatechin gallate, epigallocatechin gallate, gallocatechin, and mixtures thereof. In one embodiment, the catechin is free of caffeine and is extracted and enriched from a green tea plant source. In a particular embodiment, the catechin is epigallocatechin gallate. Various purified forms of catechins are commercially available from Sabinsa (Piscataway, N.J.), Active Organics (Lewisville, Tex.), and Arch Personal Care Products (South Plainfield, N.J.). In one embodiment, the green tea extract is colorless and devoid of tannins and other color impurities.

The catechin mixture may be present in an amount of from about 0.0001% to about 50% by weight of the composition, in another embodiment from about 0.001% to about 10%, in yet another embodiment from about 0.01% to about 5%, and in yet another embodiment from about 0.1% to about 2.5%.

J. Phytosterols

The hair care compositions of the present invention may comprise an effective amount of one or more phytosterols, including but not limited to those selected from the group consisting of β-sitosterol, campesterol, brassicasterol, Δ5-avennasterol, lupenol, α-spinasterol, stigmasterol, their derivatives, and combinations thereof. In one embodiment, the phytosterol is selected from the group consisting of β-sitosterol, campesterol, brassicasterol, stigmasterol, their derivatives, and combinations thereof.

Phytosterols of the present invention can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. In one embodiment, the phytosterols are free sterols. As used herein, “phytosterol” includes isomers, tautomers, and derivatives (e.g., esters) of such and are commercially available from Aldrich Chemical Company (Milwaukee, Wis.), Sigma Chemical Company (St. Louis, Mo.), Cognis, and Karlshamns (Karlshamns, Sweden).

Phytosterol may be present in an amount of from about 0.01% to about 50% by weight of the composition, in another embodiment from about 0.1% to about 20%, in yet another embodiment from about 0.2% to about 15%, and in yet another embodiment from about 0.5% to about 10%.

K. Panthenol and Pantothenic Acid Derivatives

The compositions of the present invention may comprise an effective amount of panthenol and/or pantothenic acid derivatives. Panthenol and its derivatives can include D-panthenol ([R]-2,4-dihydroxy-N-[3-hydroxypropyl)]-3,3-dimethylbutamide), DL-panthenol, pantothenic acids and their salts, preferably the calcium salt, panthenyl triacetate, royal jelly, panthetine, pantotheine, panthenyl ethyl ether, pangamic acid, pantoyl lactose, Vitamin B complex, or mixtures thereof.

Compositions comprising pantothenic acid derivatives that remain more stable than panthenol and other similar materials in acidic compositions or in compositions containing acid-producing materials such as aluminum-containing actives, can also be suitable for use herein. The selected pantothenic acid derivatives are most typically in liquid form and dispersed throughout or otherwise solubilized within the liquid carrier component of the composition.

The term “pantothenic acid derivative” as used herein refers to those materials that conform to the formula: 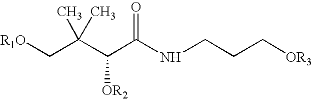
wherein R₁, R₂ and R₃ are hydrogen, C₂-C₂₀ hydrocarbons, C₂-C₂₀ carboxylic acid esters, or combinations thereof, provided that not more than two of R₁, R₂ and R₃ are hydrogen. In one embodiment, R₁, R₂ and R₃ are independently selected from hydrogen, C₂-C₈ hydrocarbons, C₂-C₈ carboxylic acid esters, or combinations thereof; in another embodiment, R₁ and R₂ are hydrogen, and R₃ is a C₂-C₈ hydrocarbon, C₂-C₈ carboxylic acid ester, or combinations thereof; in yet another embodiment, R₁ and R₂ are hydrogen and R₃ is ethyl. The selected pantothenic acid derivatives may be derived or otherwise obtained from any known source, which may include pantothenic acid or materials other than pantothenic acid, so long as the resulting material has the above defined chemical formula.

Specific non-limiting examples of selected pantothenic acid derivatives for use herein include ethyl panthenol, panthenyl triacetate, and combinations thereof. In a particular embodiment, a pantothenic acid derivative comprises the d-isomeric form(s) of such derivative form(s), such as d-ethyl panthenol.

In one embodiment, the panthenol and/or pantothenic acid derivative is used in an amount of from about 0.01% to about 10%, in another embodiment from about 0.1% to about 5%, and in still another embodiment from about 0.2% to about 3%, by weight of the composition.

L. Spin Trap Nitrones

The hair care compositions of the present invention may comprise an effective amount of at least one nitrone derivative. Nitrones are capable of irreversibly capturing electrons and/or free radicals, thereby reducing the relative amount of oxidative potential in a microenvironment. Thus, these materials have been referred to as “spin traps” since the ability to detect a free radical via spectroscopic means involves monitoring the spin resonance of free radicals. By irreversibly binding the free radical, the spectroscopic signal becomes reduced due to the free radical becoming trapped by a nitrone such as ♀-phenyl butyl nitrone (PBN). These can include α-phenyl butyl nitrone (PBN), PBN doxylcyclohexane radicals, 5,5-dimethylpyrroline N-oxide (DMPO), α-(4-pyridyl 1-oxide)-N-tert-butylnitrone (POBN), 2,2,6,6-tetramethylpiperidine 1-oxide, 4-hydroxytetramethylpiperidine 1-oxide, and the salts of N-(1-oxido-2,2,6,6-tetramethyl-4-piperidyl)-N,N-dimethyl-N-hydroxyethylammonium, 3,5-dibromo-4-nitrosobenzenesulfonic acid, 2-methyl-2-nitrosopropane, nitrosodisulfonic acid, α-(4-pyridyl-1-oxide)-N-t-butylnitrone, 3,3,5,5-tetramethylpyrroline N-oxide, and 2,4,6-tri-t-butylnitrosobenzene, or spin-trapping derivatives thereof, and mixtures thereof. In a particular embodiment, the spin trap is PBN.

M. Ursolic Acid

The hair care compositions of the present invention may comprise an effective amount of ursolic acid. Ursolic acid of the present invention can be synthetic or natural in origin and can be used as essentially pure compounds or mixtures of compounds (e.g., extracts from natural sources). Ursolic acid is commercially available from such suppliers as Sabinsa (Piscataway, N.J.) and Crodarom S.A.S. (Chanac, France). Ursolic acid may be present in an amount of from about 0.0001% to about 50% by weight of the composition, in one embodiment from about 0.001% to about 10%, in another embodiment from about 0.01% to about 7.5%, and in yet another embodiment from about 0.05% to about 5%.

N. Compounds Derived from Plant Extracts

The hair care compositions of the present invention may comprise an effective amount of compound(s) derived from plant extract(s), including but not limited to those selected from the group consisting of leguminosae, solanaceae, gramineae, and cucurbitaceae. In one embodiment, the compound derived from plant extracts is a protease inhibitor and one or more isoflavones. Isoflavone examples include genistein and daidzein. Compounds derived from plant extracts may be present in an amount of from about 0.0001% to about 50% by weight of the composition, in one embodiment from about 0.001% to about 10%, in another embodiment from about 0.01% to about 7.5%, and in yet another embodiment from about 0.05% to about 5%.

Natural plant extracts useful herein can include compounds extracted from any part of the plant of saw palmetto, willow herb, pumpkin seed, creosote, sea-buckthorn oil, capsicum, Echinacea angustifolia, Echinacea purpurea, Lithosperumum, Rosaceae, Sanguisorba officinalis, Tropaeolum majus, white birch and rubiaceae plant groups, Juniperus genus, malt, from genus Centipeda, Cinnamonum verum, Curcurbita pepo, Epilobium roseum, Salvia officinalis, Cassia obtusifoila Linne, Pleione genus, Curcuma longa, Salix alba, Hamamelis virginiana, Diopyros kaki, Hydrangea macrophylla, Hydrangea serrata, Iridaceae genus, Moraceae Humulus, Ikurinin, Regulo plant (Abelmoschus moschatus), Wolo plant (Borassus flabellifer), Hedera helix, Lithospermu, Scutellaria genus, tomato, Commiphora myrrha, Cymbopogon nardus, Lagerstroemia speciosa, Phyllanthus nuriri, Smilax zeylanica, Woodfordia fruticosa, Cistanche salsa, Larrea divaricata, Plantago asiatica, Stachys sieboldii, lavender, lemon, carrot, ginger, clove, honey, juniper, almond, palmarosa, eucalyptus, rosemary, sugars, Coix lachryma-jobi, bur marigold infusion, bacterium ribosomes, conifer extract, daisy infusion, tea tree oil, spearmint, honey, ant eggs, Bowman Birk inhibitor, peach oil, essential oils, aloe, elasatin decomposition enzyme inhibitor, peptides, plant fruit enzymes, shogaol, zingerone, zingiberol, zingiberone, and combinations thereof.

O. Metabolic Modulators

Hair care compositions herein can include an effective amount of a metabolic modulator. Metabolic modulators useful herein include 5′-p-fluorosulphonyl benzoyl adenosine, 5-keto-D-fructose, 5-keto-D-fructose-1,6-bisphosphate, 6-amino-6-deoxy-glucose, inhibitor of a cysteine pathway enzyme, guanidino succinic acid, cysteine sulphinic acid, phosphoglycerate, cysteamine, cysteine sulphinic acid, cysteinyl-glycine, D-cysteine, inhibitor of a cholesterol pathway enzyme, inhibitor of the formation of glycoproteins, N-acetylcysteine (NAC), D-mannosamine, N-alpha-(p-tosyl)-L-lysine chloromethyl ketone, N-acetyl-beta-D-mannosamine, oxaloacetic acid, finasteride, arginase inhibitor, N-phosphonoacetyl-aspartic acid, N-alpha-acetyl-L-arginine, N-alpha-benzoyl-L-argininamide, N-alpha-benzoyl-L-arginine, N-alpha-benzoyl-L-arginine methyl ester, NG-L-arginine benzyl ester, NG-nitro-L-arginine, NG-nitro-L-arginine methyl ester, dithiothreitol, glutathione, homocysteine, lipoic acid, 2-mercaptoethanol, mercaptopropionic acid, thiodiglycol, thiodiglycolic acid, thioglycerol, thioglycolic acid, thiolactic acid, thiomalic acid, thiopropionic acid, thiosalicylic acid, thioxanthine, H-homoarginine, L-alanosine, L-argininamide, L-asparaginamide, L-cysteine methyl ester, alpha-methyl-DL-methionine, dimethyl cysteamine, sulfotransferase inhibitors, N(G)-methyl-L-arginine, alpha.-fluoromethylhistidine, inhibitors of glutamine metabolism, glutathione synthesis stimulators, fatty acids, chelating agents, pravastatin, rivastatin, simvastatin, squalestatin, fluvastatin, mevastatin, mevinolin, lovastatin, cysteine, chlorotaurine, 2-mercaptopropionic acid, diethyldithiocarbamic acid, aromatase inhibitors, glutathione S-transferase modulators, peptide or trisamine carrying fatty acid ester and dithioalkanoyl groups, sorbic acid, vitamin K, vitamin F, phloretin, and combinations thereof.

P. Anti-Proliferatives

The hair care composition of the present invention can include an effective amount of an anti-proliferative. Anti-proliferatives useful herein include difluoromethylornithine (DFMO), methacycline, protein kinase C inhibitors, smoothened inhibitors, protein-tyrosine kinase inhibitors, tyrphostins and tryphostins, cyclooxygenase inhibitors, 5-alpha-reductase inhibitors, adenylsuccinate synthetase inhibitor, aspartate transcarbamylase inhibitor, gammaglutamyl transpeptidase inhibitor, ornithine decarboxylase inhibitors, non-steroidal anti-inflammatory drugs (NSAIDS), lipoxygenase inhibitors and stimulants, nordihydroguaianetic acid (NDGA), inhibitor of alkaline phosphatase, doxycycline, minocycline, taxodione, taxodone, bacteriostatic or haemostyptic agent, especially stannous fluoride, alpha-ethyl-ornithine, nalidixic acid, tetracycline, inhibitors of the hypusine biosynthetic pathway, methacycline, methylglyoxal bis(guanylhydrazone), bromocryptine, trihydroxypurine, etoposide, guanidino succinic acid, matlystatin-B, 5′-deoxy-5′-(N-methyl-N-(2-aminooxy-ethyl)-aminoadenosine (MAOEA), 5′-deoxy-5′-methyl-thioadenosine, doxycycline, pyrimidine-cyanoguanidine derivatives, substituted amidine or guanidine, butyric acid derivatives, hydroxamic acid and analogues, medroxyprogesterone acetate, megestrol acetate, melengestrol acetate, nomegestrol acetate, mycophenolic acid, cyanoguanidine derivatives, diethyl glyoxal bis(quanylhydrazone), and combinations thereof.

Q. Signal Transduction Modulators

An effective amount of a signal transduction modulator can be included in the hair care composition of the present invention. Signal transduction modulators useful herein include Br-cAMP, E6AP-binding polypeptides, ethoxyquin, anti-angiogenic steroids, CDK binding proteins, chimeric polypeptide with cyclin-dependent kinase (CDK) binding motif, suppressor of angiogenesis, alpha- or gamma-linolenic acid, EGF and analogues. Hairless protein and analogues, estrogen agonists or antagonists, proteoglycans or glycosaminoglycans, phytoestrogen, hedgehog antagonists, patched antagonists, interleukin-1 antagonist, alpha-TNF antagonist, leuteinizing hormone-releasing hormone and analogues, GnRH inhibitors, Heptapeptide luteinizing hormone releasing hormone (LHRH) analogs, 1-halomethyl-5alpha-androstanes and delta-androstenes, 3-oxo-4-aza-5 alpha-androstane derivatives, finasteride, spironolactone, propyl gallate, eicosapentaenoic acid, lavendustin A, activin A, androgen receptor blockers, quercetin, protocatechuic acid and aldehyde, methyl caffeate, apigenin, caffeic acid, progestins and antiprogestins, vitamin D and analogues including previtamin D and provitamin D, androstenedione analogues, lipoxydase, spironolactone, cyproterone acetate, progesterone, melatonin, and mixtures thereof.

R. Proteases and Protease Inhibitors

An effective amount of a protease and/or protease inhibitor can be included in the hair care composition of the present invention. Protease and protease inhibitors useful herein include 1,10-phenanthroline, elastase inhibitors, papain, trypsin and analogues, chymotrypsin, pepsin, bromelain, ficin, pancreatin, marimistat, and combinations thereof.

S. Others

An effective amount of any other appropriate hair growth inhibiting compound can be included in the hair care composition herein. Other hair growth inhibiting compounds useful herein can include phlondrin, agaric acid, vemolepin, D-penicillamine, ethacrynic acid, eupacunin, euparotin acetate, diethylaminomalonate, protocatechuic aldehyde, non-elastomeric polyolefin resin, partially fluorinated polyolefin resin, quinaldic acid, 1,8-diaminooctane, 2-methyl-6-heptyne-2,5-diamine, 3-carboxypropyl disulphide, 5-(N-benzyloxycarbonyl)-1-phenylalanamidomethyl)-3-bromo-4,5-dihydroisoxazole, 6-heptyne-2,4-diamine, actinonin, batimistat, captopril, diethyl aminomalonate, diethyldithiocarbamic acid, estramustine, ethacrynic acid, meso-dimercaptosuccinic acid, N-[N[((R)-1-phosphonopropyl)-(S)-leucyl]-(S)-phenylalanine-N-methylamide, N-phosphonalkyl dipeptides, oxaloacetic acid, phosphocysteamine, S-carbamyl-L-cysteine, S-trityl-L-cysteine, sulphasalazine, thiosalicylic acid, tyramine, 2-difluoromethyl-, 2,5-diamino pentanoic acid, herbimycin, HNMPA (AM)3, O-p-nitrohydroxylamine, cromoglycate, quinoline-3-carboxamide, 16 alpha- or beta-substituted 4-aza-5 alpha-androst-1-en-3-ones, 2-aryl-indole derivatives, 2-phenyl-3-aminoalkyl-indole derivatives, 5 alpha-androstan-3-ones, 5-(aminocarbonylalkyl)-3-(heterobicyclyl-alkylaminoalkyl)-2-phenylindole derivatives, 6-azaindole derivatives, 7-azaindole derivatives, aryl-imidazo-pyridines, carboxyalkylamine derivatives, malonamide derivatives, 2-indole carboxylic acid derivatives, aminopropanes, diethylenediamines, histamine antagonist, phenothiazines, tetrazolyl-benzofuran carboxamides, tetrazolyl-benzothiophene carboxamides, 17alpha-hydroxy-4,9(11)-pregnadiene-3,20-dione derivatives, benzothiophene derivatives, (−) cis 6(S)-phenyl-5(R)-[4-(2-pyrrolidin-1-yl ethoxyphenyl]-5,6,7,8-tetrahydronaphthelen-2-ol D-tartrate (I), tetrahydronaphthalene derivatives, tetrahydroisoquinolines, tetrahydroisoquinoline derivatives, tetrahydroisoquinoline derivatives, 3-(anilinomethylene) oxindole derivatives, benzo-[f]-quinolin-3-one derivative, ((S-(−)-N-(alpha-ethylbenzyl)-3-hydroxy-2-phenylquinoline-4-carboxamide), 24-ethyl-(delta)-4,22-cholestadien-3-one, benzoic acid lactone ether, copper, iron, zinc, 1 dehydromelengestrol acetate, 1-dehydromegestrol acetate, chlormadinone acetate, cyproterone acetate, hydrindanes, diazo compounds, tetrahydroisoquinolines, tetrahydronaphthalenes, 3-amino-2,3-dihydro benzoic acid, 6-fluoro-2,5-diamino hexanoic acid, (S)-2-amino-4-amino oxy-butyric acid, triarylmethane compounds, perfluoro-substituted aniline derivatives, 17alpha-propyltestosterone, 4-androstene-3-one 17beta-carboxylic acid, (4R)-5,10-seco-19-norpregna 4,5-diene-3,10,20-trione, chlormadinone acetate, 2-substituted 6-tetrahydronaphthyl or indanyl naphthalene derives, 2-phenyl-benzothiophene derivatives, 2-arylimino-oxaza or thiaza heterocyclic compounds, indole derivatives, dormant cell extracts, N-substituted benzyl- or thienylmethyl-4-pyridone compounds, (1H)-benzo(c)quinolizin-3-one derivatives, citric acid, Dead Sea salts, visaborol, chlorophenol, o-phenylphenol, phenol, niphtolide, 2-amino-5-substituted benzophenone, aniline derivatives, camphor oil, citric acid, conjugate comprising active agent substituted with amino acid, 11-beta-aryl-17-spiro-pyrrolin-2-ylidene N-oxide steroid, phenyl imidazolidines, coumarin derivatives, borneol, cineole, linalool, methyl heptenone, thiomolybdate compound, trifluoroanilide derivatives, and mixtures thereof.

II. Method for Maintaining a Longer Lasting Hair Style Appearance

The present invention also provides methods for maintaining a longer lasting hair style appearance. In one aspect, the method comprises applying a hair care composition comprising an effective amount of a hair growth inhibiting agent to a skin surface from which a region of styled hair grows. For instance, the hair care composition can be applied to the scalp and/or face (e.g., beard or moustache area). In another embodiment, the method comprises topically applying a hair care composition comprising an effective amount of a hair growth inhibition agent to a skin surface from which a region of styled hair grows, on a mammal seeking to reduce the frequency of hair style maintenance. In a particular embodiment, the invention provides use of a hair care composition comprising a hair growth inhibition agent for maintaining a longer lasting hair style appearance.

The region of styled hair can be located on any part of the body. For instance, it can grow from a skin surface located on at least a portion of the scalp or the face or the neck. Furthermore, the styled hair can be any desired length. For instance, the styled hair can be less than about 30 centimeters in length, less than about 15 centimeters in length, less than about 10 centimeters in length, less than about 5 centimeters in length, less than 1 centimeter in length, greater than 0.5 cm, greater than 1 cm, greater than about 5 cm, greater than about 10 cm, greater than about 15 cm, or greater than about 30 cm in length. As used herein, “styled hair” includes both hair that has already been styled, as well as hair that is intended to be styled after application of the hair growth inhibition agent composition.

In still another embodiment, the method comprises applying the composition according to a regimen, wherein said regimen comprises:

(a) cleansing the scalp and/or face to form a cleansed scalp and/or face;

(b) topically applying the composition to said cleansed scalp and/or cleansed face.

III. Article of Commerce and Method of Marketing

In another aspect, the present invention provides articles of commerce and methods of marketing hair care compositions that can be used to help maintain a longer lasting hair style appearance. In one embodiment, the article of commerce comprises:

(1) a container;

(2) a hair care composition contained within said container, wherein said hair care composition comprises a hair growth inhibition agent; and

(3) a communication, wherein said communication communicates that use of said hair care composition can help maintain a longer lasting hair style appearance.

In another aspect, the present invention provides methods of marketing hair care compositions that can be used to help maintain a longer lasting hair style appearance. In one embodiment, the method comprises:

(a) offering for sale a hair care composition comprising a hair growth inhibition agent;

(b) communicating that said composition can be used to help maintain a longer lasting hair style appearance.

In another aspect, the invention provides a marketing method that utilizes a comparison of a first hair care composition to a second hair care composition, in order to market the first hair care composition. In one embodiment, the method comprises offering for sale a first article of commerce, wherein said first article of commerce comprises:

(a) a first hair care composition; and

(b) a communication, wherein said communication compares said first hair care composition to a second hair care composition, wherein said second hair care composition is comprised in a second article of commerce, wherein said second article of commerce comprises:
(1) said second hair care composition comprising a hair growth inhibition agent; and
(2) a second communication to a potential consumer, wherein said second communication communicates that said second hair care composition can be used to help maintain a longer lasting hair style appearance.

In another aspect, the invention provides a marketing method that utilizes at least one visual cue to communicate that a first hair care composition is similar to or the same as a second hair care composition, in order to market the first hair care composition. In one embodiment, the visual cue comprises a message. In particular embodiments, the message can comprise words such as “compare,” “compare to”, “like”, “similar”, “try instead of,” or the like. In another embodiment, the visual cue can comprise the same or similar graphics as those included on or near the packaging of the second hair care composition. A visual cue can be located at or on any suitable location. For instance, a visual cue can be located on or near product packaging, or on or near a store shelf.

In a particular embodiment, the first hair care composition is marketed in a container having at least two of the same colors as the container in which the second hair care composition is marketed. In one embodiment, the method comprises a method of marketing a first hair care composition, wherein said method comprises:

(a) offering for sale a first article of commerce, wherein said first article of commerce comprises:
(1) a first container;
(2) a first hair care composition contained within said container;
(3) a first set of graphics disposed upon said first container, wherein said first set of graphics comprises at least two colors;

(b) locating said first article of commerce within visual sight of a second article of commerce, wherein said second article of commerce comprises:
(1) a second container;
(2) a second hair care composition contained within said second container,
(3) a second set of graphics disposed upon said second container, wherein said second set of graphics comprises:
   (i) at least two of the same colors as those colors included in said first set of graphics; and
   (ii) a communication to a potential consumer, wherein said communication informs said potential consumer that said second hair care composition can be used to help maintain a longer lasting hair style appearance.

As used herein, the term “potential consumer” means an actual or potential purchaser and/or an actual or potential user of the article of commerce and/or hair care composition.

Any container from which the hair care composition can be stored and/or contained can be used herein. Suitable containers can include, but are not limited to, bottles, tottles, tubes, pouches, boxes, tubs, and cans. Furthermore, containers can include primary containers, which contain the hair care composition itself, or secondary containers, which contain at least one primary container that contains the composition.

As used herein, “set of graphics” or “graphics” refers to the text and/or pictorial images that are disposed on a container. As used herein, “disposed on” means integral with and/or located on the container and can include, but is not limited to, disposed directly thereon (e.g., printed directly on the container), disposed indirectly thereon (e.g., printed on a sticker that is affixed to the outer portion of the container), and/or applied to the container by any other suitable means (e.g., sprayed, bonded, drawn, painted, printed, or molded).

As used herein, “communication” means a message, and can include but is not limited to a printed (e.g., printed material attached directly or indirectly to the container), electronic, or broadcast message.

Optionally, said first article of commerce and said second article of commerce can be located within visual sight of one another. In a particular embodiment, said first article of commerce and said second article of commerce can be located adjacent to one another on a retail shelf or other retail display.

As used herein, “located within visual sight of one another” means that the first article of commerce and the second article of commerce are located in proximity to one another such that a human with unassisted 20/20 vision can see both the first article of commerce and the second article of commerce at the same time. In a particular embodiment, said first article of commerce and said second article of commerce are located within 2 meters of each other. In another embodiment, said first article of commerce and said second article of commerce are located within 1 meter of each other. In a specific embodiment, said first article of commerce and said second article of commerce are located within 0.5 meter of each other.

As used herein, “similar” means alike in someway. For instance, alike in composition, composition of active ingredients, and/or benefits that can be provided from use of the composition.

### EXAMPLES

The following are non-limiting examples of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

### Examples 1-4

| | 1 | 2 | 3 | 4 |
|---|---|---|---|---|
| Component | (wt %) | (wt %) | (wt %) | (wt %) |
| Water | Q.S. | Q.S. | Q.S. | Q.S. |
| Rheology modifying system, anionic | 0.0500 | 0.0500 | — | — |
| polymer MVE/MA crosslinked copolymer | | | | |
| (Stabileze 06) | | | | |
| Rheology modifying system, clay Hydrous | 0.0500 | 0.0500 | 0.05 | — |
| Na, Mg silicate (Laponite XLS) | | | | |
| Hydroxypropyl methylcellulose (PrimaFlo) | — | — | 0.1 | — |
| Polyquaternium 10 (Ucare Polymer LR- | 0.5000 | 0.5000 | 0.5 | 0.5000 |
| 400) | | | | |
| Coconut monoethanolamide (Monamid | 1.0909 | 1.0286 | 1.0286 | 1.5000 |
| CMA) | | | | |
| Disodium EDTA (Disslovine Na₂S) | 0.1400 | 0.1400 | 0.14 | 0.0991 |
| Sodium Benzoate (Purox S Grains) | 0.2500 | 0.2500 | 0.25 | 0.2500 |
| Sodium Citrate Dihydrate | 0.4520 | 0.4520 | 0.452 | 0.4520 |
| Sodium Laureth-3- Sulfate (SLE3S) | 2.1818 | — | — | — |
| Cocamidopropyl Betaine (Tegobetaine F- | 2.1818 | — | — | — |
| B) | | | | |
| Sodium lauryl sulfate (SLS) | 6.5455 | — | — | — |
| Citric Acid | 0.0781 | — | — | 0.0400 |
| BHT | 0.5000 | 0.5000 | 0.5000 | 0.5000 |
| Sodium Chloride | 0.2500 | 0.7500 | 0.50 | 0.0145 |
| Sodium Hydroxide | 0.0126 | — | — | — |
| Dimethicone (Viscasil 3000,000) | 1.3510 | 1.3510 | 1.3510 | 1.3510 |
| Ammonium Laureth-3-Sulfate (AE3S) | 0.0676 | 4.1143 | 6.00 | 6.0000 |
| Ethylene glycol distearate (EGDS) | 1.5000 | 1.5000 | 1.5 | 1.5000 |
| Ammonium Lauryl Sulfate (ALS) | 1.5000 | 6.8751 | 6.8751 | 10.0000 |
| Hexamidine diisethionate | 0.1000 | 0.1000 | 0.1000 | 0.1000 |
| Glyceryl dilaurate | 2.0000 | 2.0000 | 2.0000 | 2.0000 |
| Methylchloroisothiazolinone & | 0.0005 | 0.0005 | 0.0005 | 0.0005 |
| Methylisothiazolinone (Kathon CG) | | | | |
| Fragrance | 0.7000 | 0.7000 | 0.7 | 0.7000 |
| PEG 7M (Polyox WSR-N-750) | 0.1000 | — | — | 0.1000 |
| DL Panthenol 50% soln. (DL-Panthenol | 0.0300 | 0.0300 | 0.03 | 0.0300 |
| 50L) | | | | |
| DL Panthenyl Ethyl Ether (Pantyl Ethyl | 0.0300 | 0.0300 | 0.03 | 0.0300 |
| Ether) | | | | |
| Lysine Monochloride | 0.0280- | 0.0280 | 0.0280 | 0.0280 |
| L-Tyrosine Methylester Hydrochloride | 0.0138 | 0.0138 | 0.0138 | 0.0138 |
| (Methyl Tyrosine) | | | | |
| Histidine | 0.0080 | 0.0080 | 0.0080 | 0.0080 |
| Cetyl Alcohol | — | — | | 0.9000 |

### Example 5-7

### Conditioner Examples

| | 5 | 6 | 7 |
|---|---|---|---|
| Component | (wt %) | (wt %) | (wt %) |
| Dimethicone compound-1 | — | 4.2 | |
| *1 | | | |
| Dimethicone compound-2 | — | — | 2.000 |
| *2 | | | |
| Silicone compound-2 *3 | 3.500 | — | |
| Behenyl trimethyl | 2.250 | — | 3.380 |
| ammonium chloride *6 | | | |
| Isopropyl alcohol | 0.598 | — | 0.900 |
| Stearamidopropyl | — | 2.000 | — |
| dimethylamine *7 | | | |
| Glutamic acid *8 | — | 0.640 | — |
| Cetyl alcohol *9 | 1.900 | 2.500 | 2.300 |
| Stearyl alcohol *10 | 4.600 | 4.500 | 4.200 |
| Polysorbate-20 *11 | — | — | — |
| PPG-34 *12 | — | — | — |
| Polyalphaolefin *13 | — | — | — |
| BHT | 0.500 | 0.500 | 0.500 |
| Benzyl alcohol | 0.400 | 0.400 | 0.400 |
| Glyceryl dilaurate | 2.000 | 2.000 | 2.000 |
| Methylchloroisothiazolinone/ | 0.0005 | 0.0005 | 0.0005 |
| Methylisothiazolinone | | | |
| *14 | | | |
| Perfume | 0.350 | 0.500 | 0.350 |
| NaOH | 0.014 | — | 0.014 |
| Panthenol *15 | 0.050 | — | 0.05 |
| Panthenyl ethyl ether *16 | 0.050 | — | 0.05 |
| Hexamidine diisethionate | 0.100 | 0.100 | 0.100 |
| Hydrolyzed collagen *17 | — | 0.010 | — |
| Vitamin E *18 | — | 0.010 | — |
| Decyl Glucoside *19 | — | — | — |
| Octyl methoxycinnamate | — | 0.090 | — |
| Benzophenone-3 | — | 0.090 | — |
| Disodium EDTA | 0.127 | 0.127 | 0.127 |
| Deionized water | Qs | Qs | Qs |
| Definitions of Components | | | |
| *1 Dimethicone/Cyclomethicone: a blend dimethicone having a viscosity of 18,000,000 mPa · s and cyclopentasiloxane available from GE Toshiba | | | |
| *2 Dimethicone blend: a blend of dimethicone having a viscosity of 18,000,000 mPa · s and dimethicone having a viscosity of 200 mPa · s available from GE Toshiba | | | |
| *3 Available from GE having a viscosity 10,000 mPa · s, and having following formula (I): (R₁)ₐG₃₋ₐ-Si—(—OSiG₂)ₙ-(-OSiG_{b}(R₁)_{2-b})ₘ-O—SiG₃₋ₐ(R₁)ₐ (I) wherein G is methyl; | | | |
| # a is an integer of 1; b is 0, 1 or 2, preferably 1; n is a number from 400 to about 600; m is an integer of 0; R₁ is a monovalent radical conforming to the general formula CqH_{2q}L, wherein q is an integer of 3 and L is —N(CH₃)₂ | | | |

*6 Behenyl trimethyl ammonium chloride/Isopropyl alcohol: Genamin KDMP available from Clariant
*7 Stearamidopropyl dimethylamine: Lexamine S-13 available from Inolex
*8 Glutamic acid: available from Ajinomoto
*9 Cetyl alcohol: Konol series available from Shin Nihon Rika.
*10 Stearyl alcohol: Konol series available from Shin Nihon Rika.
*11 Polysorbate-20: Glycosperse L-20K available from Lonza Inc.
*12 PPG-34: New Pol PP-2000 available from Sanyo Kasei.
*13 Polyalphaolefin: PureSyn 100 available from ExxonMobil Chemical Company
*14 Methylchloroisothiazolinone/Methylisothiazolinone: Kathon CG available from Rohm & Haas
*15 Panthenol: Available from Roche.
*16 Panthenyl ethyl ether: Available from Roche.
*17 Hydrolyzed collagen: Peptein 2000 available from Hormel.
*18 Vitamin E: Emix-d available from Eisai.
*19 Decyl glucoside: Plantacare 2000UP available from Cognis Japan Ltd.

### Example 8

| | 8 |
|---|---|
| Component | (wt %) |
| Alcohol 100% DEB 100 (Ethanol) | 25.000 |
| Carbomer (Carbopol Ultrez 10) | 0.100 |
| Hexamidine diisethionate | 0.100 |
| Glyceryl dilaurate | 2.000 |
| BHT | 0.500 |
| Triethanolamine | 0.200 |
| Caffeine | 1.500 |
| Niacinamide | 5.000 |
| Panthenol | 0.300 |
| Deionized water | Qs |

### Example 9

| | 9 |
|---|---|
| Component | (wt %) |
| Propylene glycol | 9.500 |
| Ammonium hydroxide | 5.000 |
| Ethoxydiglycol | 4.000 |
| Ethanolamine | 4.500 |
| Oleic acid | 1.000 |
| Hexylene glycol | 6.000 |
| Hexamidine diisethionate | 0.100 |
| Glyceryl dilaurate | 2.000 |
| BHT | 0.500 |
| Cocamidopropyl betaine | 3.500 |
| Oleth-10 | 0.300 |
| Oleth-2 | 0.300 |
| Dilinoleic acid | 1.500 |
| C12-C15 Pareth-3 | 0.500 |
| Soytrimonium chloride | 7.000 |
| Sodium metasilicate | 0.050 |
| Erythorbic acid | 0.500 |
| EDTA | 0.030 |
| Sodium sulfite | 0.300 |
| 1-Phenyl-3-methyl-5-pyrazolone | 0.200 |
| Deionized water | Qs |

### Example 10

| | 10 |
|---|---|
| Component | (wt %) |
| Ethanol | 51.800 |
| Propylene glycol | 5.000 |
| Propellant P75 | 4.300 |
| Cetyl alcohol | 2.200 |
| Glyceryl dilaurate | 2.000 |
| Stearyl alcohol | 1.000 |
| Polyoxyethylene lauryl alcohol | 1.000 |
| BHT | 0.500 |
| Polysorbate 60 | 0.400 |
| Hexamidine diisethionate | 0.100 |
| Acetic acid | Qs pH 6.0 |
| Deionized water | Qs |

### Example 11

### Method of Marketing

The shampoo of Example 1 is packaged into a blue and white container and offered for sale to consumers at a retail store. A label on the container communicates that when this shampoo is used to wash color-treated hair, the frequency at which the consumer will need to re-color his hair to cover the growth of the roots will be reduced.

### Example 12

### Method of Marketing

A shampoo contained in a blue and white bottle (herein “Subject Shampoo”) is located on a shelf next to the shampoo of Example 11. A label is attached to the Subject Shampoo's bottle which directs the consumer to compare the Subject Shampoo to the shampoo of Example 11.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited herein are incorporated herein by reference in their entirety, except to the extent that any part of such documents conflict with the teachings of the present application; the citation of any document is not to be construed as an admission that it is prior art with respect to the present invention.

What is claimed is:

1. A method of marketing a hair care composition that can be used to help maintain a longer lasting hair style appearance, wherein said method comprises:
(a) offering for sale a hair care composition comprising a hair growth inhibiting agent;
(b) communicating that said composition can be used to help maintain a longer lasting hair style appearance.

2. A method of marketing a first hair care composition, wherein said method comprises:
(a) offering for sale a first hair care composition;
(b) communicating that said first hair care composition is comparable to a second hair care composition when used to help maintain a longer lasting hair style appearance, wherein said second hair care composition comprises a hair growth inhibiting agent.

3. A method of marketing a first hair care composition, wherein said method comprises:
(a) offering for sale a first article of commerce, wherein said first article of commerce comprises:
(1) a first container;
(2) a first hair care composition contained within said container;
(3) a first set of graphics disposed upon said first container, wherein said first set of graphics comprises at least two colors;
(b) locating said first article of commerce within visual sight of a second article of commerce, wherein said second article of commerce comprises:
(1) a second container;
(2) a second hair care composition contained within said second container,
(3) a second set of graphics disposed upon said second container, wherein said second set of graphics comprises:
(i) at least two of the same colors as those colors included in said first set of graphics; and
(ii) a communication to a potential consumer, wherein said communication communicates to said potential consumer that said second hair care composition can be used to help maintain a longer lasting hair style appearance.

4. A method of marketing a first hair care composition, wherein said method comprises offering for sale a first article of commerce, wherein said first article of commerce comprises:
(a) a first hair care composition; and
(b) a communication, wherein said communication compares said first hair care composition to a second hair care composition, wherein said second hair care composition is comprised in a second article of commerce, wherein said second article of commerce comprises:
(1) said second hair care composition comprising a hair growth inhibition agent; and
(2) a second communication to a potential consumer, wherein said second communication communicates that said second hair care composition can be used to help maintain a longer lasting hair style appearance.

5. A method for maintaining a longer lasting hair style appearance, wherein said method comprises topically applying a hair care composition that comprises an effective amount of a hair growth inhibiting agent to the scalp or neck or face of a mammal seeking to maintain a longer lasting hair style appearance.

6. The method of claim 5, wherein said method comprises applying said composition according to a regimen, wherein said regimen comprises:
(a) cleansing the scalp or neck or face to form a cleansed scalp or neck or face; and
(b) topically applying said composition to said cleansed scalp or neck or face.

7. An article of commerce, wherein said article of commerce comprises:
(1) a container;
(2) a hair care composition contained within said container, wherein said hair care composition comprises a hair growth inhibition agent;
(3) a communication, wherein said communication communicates that use of said hair care composition can help maintain a longer lasting hair style appearance.

8. The article of claim 7, wherein said hair growth inhibition agent is selected from the group consisting of butylated hydroxytolune, butylated hydroxyanisole, glyceryl dilaurate, hexanediol, panthenol, agmatine, aminoguanidine, phenyl butyl nitrone and other spin traps, ethoxyquin, hexamidine, hexyl isobutyrate, menthyl anthranilate, methofuran, 3-butylidenepthalide, cetyl pyridinium chloride, green tea extract, catechins, phytosterols, ursolic acid, plant extracts, plant extract compounds, 3-butylidenepthalide, its salts, its derivatives, and mixtures thereof, and combinations thereof.

9. The article of claim 8, wherein said longer lasting hair style appearance reduces the frequency of hair style maintenance, and wherein said hair style maintenance is selected from the group consisting of hair coloring, hair cutting, hair shaving, hair trimming, permanent hair curling, permanent hair waving, permanent hair relaxing, permanent hair straightening, and combinations thereof.

10. The article of claim 9, wherein said hair growth inhibiting agent comprises hexamidine.
